(19) United States
(12) Patent Application Publication
(10) Pub. No.: US 2019/0117062 A1
(43) Pub. Date: Apr. 25, 2019

(54) OCULAR VERGENCE-ENABLED EYE TRACKING METHODS AND SYSTEMS

(71) Applicants: Virginia Commonwealth University, Richmond, VA (US); CooperVision International Holding Company, LP, St. Michael (BB)

(72) Inventors: Woon-Hong Yeo, Suwanee (GA); Saswat Mishra, Henrico, VA (US); Sarah Tao, Pleasanton, CA (US); Arthur Back, Danville, CA (US)

(21) Appl. No.: 16/162,476

(22) Filed: Oct. 17, 2018

Related U.S. Application Data
(60) Provisional application No. 62/574,376, filed on Oct. 19, 2017.

(51) Int. Cl. A61B 3/113; G06F 3/01; A61B 5/0496; A61B 5/00; G02B 27/00

(52) U.S. Cl. CPC A61B 3/113; G06F 3/013; G06K 9/00604; A61B 5/6814; G02B 27/0093; A61B 5/0496

(57) ABSTRACT

Methods and systems for tracking ocular vergence movements of an eye of a person are described. The methods and systems involve placement of flexible/stretchable skin-like electrodes on a person's head and recording the biopotential or electrooculogram of the person as the eye or eyes move to different focal positions, such as near distances, intermediate distances, and far distances. A data acquisition unit can record the electrooculogram and transmit the electrooculogram data to a computer for classification or for further processing. The data can be used with ophthalmic devices to provide a visual change for a person with the ophthalmic device.

### BACKGROUND OF THE INVENTION

### Field of the Invention

The present invention generally relates to eye tracking methods and systems, and more particularly, to ocular vergence-enabled gaze tracking methods and systems to indicate the position of an eye or eyes of a person at different focal distances, which can be used to provide a signal or signals to one or more ophthalmic lenses having an electronically adjustable optic, among other things.

### Background Description

Eye tracking systems have been described for people in fields of rehabilitation, activity tracking, and gaming. For example, infrared (IR) camera based eye tracking systems exist.

Vergence and accommodation are the yin and yang of eye focus that allows humans to focus and distinguish objects at various distances. In accommodation, the ciliary muscles control the zonules, which controls the crystalline lens in the eye, thereby modifying the focal distance of light on the retina. Many groups in the 1970s had studied ocular accommodation using electroencephalography. These accommodative studies, demonstrated alpha rhythms arising in test subjects that voluntarily allow objects to focus and defocus. An extended application of this functionality was used by the military for Morse code translation. The focus and defocus functionality makes accommodative measurements better at distinguishing visual fatigue via occipital lobe measurements. To this date, it is believed that no accommodative study has shown multi-class distinct spatial changes via electro-potential studies. Vergence motions are opposing movements of the eye in order to conform to binocular vision. These motions are relatively difficult to measure with a non-invasive contact method, such as methods using capacitive bioelectrodes.

Wearable electronics are increasing in use. Flexible skin-like electrodes have made their way into previous electrophysiological measurement systems and methods, such as, electromyograms (EMG), electroencephalograms (EEG), electrocardiograms (ECG), and more recently electrooculograms (EOG). These skin-like electronics reduce issues of rigidity associated with conventional electronic recording devices, and reduce the use of conductive gels for biopotential recordings, among other things. Many conductive materials have been used in these systems for skin recordings such as silver, gold, copper, and more recently carbon based electrodes.

Contact lenses have been described that include adjustable optics that are intended to be effective in changing the refractive properties of the contact lenses. For example, contact lenses containing electronic components have been described as new devices which may be helpful in improving visual acuity of presbyopes, who suffer from a reduced ability to accommodate.

There remains a need for improvements in eye tracking methods and systems. In addition, there remains a need for new methods and systems to enable ophthalmic devices to adjust their focal properties based on the viewing distance a person is gazing.

### SUMMARY OF THE INVENTION

According to the present disclosure, there are presented alternatives to conventional EOG recordings, bulky hydrogel electrodes, rigid telemetric devices, and classification algorithms. Altering the electrode positioning is difficult with hydrogel or conductive gel electrodes but it is rather facile with skin-like electrodes. Skin-like electrodes conform well to the skin and demonstrate low impedances for data recordings on irregular locations, such as the nose. Among other things, embodiments of the invention implement an integrative system with the skin-like electrodes for continuous data recordings wirelessly. In addition, skin-like electrodes have enabled the recordings of vergence motions, and machine learning classifiers are used to verify the real-time observation of objects in depth, which is useful in confirming the focal distance a person is viewing. A real-time classification algorithm is used with the vergence motions to demonstrate activity recognition feasibility.

As discussed herein, aspects of the present invention relates to methods and systems for tracking ocular vergence movements of an eye of a person. The methods and systems involve placement of flexible skin-like electrodes on a person's head and recording the biopotential or electrooculogram of the person as the eye or eyes move to different focal positions, such as near distances, intermediate distances, and far distances. A data acquisition unit can record the electrooculogram and transmit the electrooculogram data to a computer for classification or for further processing. The data can be used with ophthalmic devices to provide a visual change for a person with the ophthalmic device.

Additional aspects and embodiments of the present invention will be apparent from the following description, drawings, and claims. As can be appreciated from the foregoing and following description, each and every feature described herein, and each and every combination of two or more of such features, is included within the scope of the present invention provided that the features included in such a combination are not mutually inconsistent. In addition, any feature or combination of features may be specifically excluded from any embodiment of the present invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing and other objects, aspects and advantages will be better understood from the following detailed description of a preferred embodiment of the invention with reference to the drawings, in which:

FIG. 1a is an image of the physical eye vergence test apparatus used in the invention made of aluminum framing, transparent Plexigas, and a laser diffraction element;

FIG. 1b is a top view of a skin-electrode and an angled view of the skin-electrode;

FIG. 1c illustrates the finite element analysis (FEA) which validates the stretchability of the electrode in the elastic region of the gold material;

FIG. 1d similarly illustrates the result of FEA that validates the bending capability of the electrode up to 180 degrees where the maximum principal strain for bending is 1/10^{th} of stretching;

FIG. 2a illustrates a conventional electrooculograms setup;

FIG. 2b illustrates ocular vergence setup one with highest accuracy and most electrodes;

FIG. 2c illustrates ocular vergence setup two with the same number of electrodes as the conventional setup as shown in FIG. 2a;

FIG. 2d is a graph showing the test apparatus degrees of motions;

FIG. 2e is a graph of eye vergence motion forwards;

FIG. 2f is a graph of eye motions backwards with corresponding resolution;

FIG. 2g illustrates eye vergence change as a user moves forwards;

FIG. 2h is a graph of vergence resolution of five subjects from group two;

FIG. 3a is a graph of a raw signal with baseline drift;

FIG. 3b is a graph of filtered and denoised signal with Butterworth bandpass and the mean subtracted;

FIG. 3c is a graph of the second order derivative filter with filtered signal;

FIG. 3d is a graph comparing the sixth order derivative and second order derivative filter for thresholding;

FIG. 3e is a graph of signal to noise ratio (SNR) of sixth order derivative better compared to second order derivative SNR so noise threshold filter can be set lower;

FIG. 3f is a graph of sixth order derivative filter with the largest signal comparable enough to filter out blinks;

FIG. 3g is a graph illustrating an example of three forward motions and corresponding sixth order derivative peaks;

FIG. 3h is a graph illustrating an ensemble subspace discriminant classifier in the feature space;

FIG. 4a is a table of features which are all compatible with Matlab coder;

FIG. 4b is a table showing cross validation of KNN, Ensemble, discriminant, and SVM;

FIG. 4c is an illustration of subjects one to five cross validation results with highest accuracy;

FIG. 4d is an illustration of subjects six to ten validation with ocular vergence of subject two;

FIG. 4e is an illustration of real-time results from subjects six to ten while mounted on test apparatus;

FIG. 4f is an illustration of real-time results from subjects four, eight and nine while dismounted;

FIG. 5a is an illustration of test apparatus dismounted with electrode setup on the face and corresponding signal from the test in one direction;

FIG. 5b is an illustration of activity recognition with smartphone, monitor and television showing the corresponding signal is slightly different in the vertical channel due to the nature of the positioning of the screens;

FIG. 6 illustrates a data acquisition setup with electrodes on the skin with signals wirelessly transferred to a tablet via Bluetooth;

FIG. 7 is a plan view of one possible contact lens which can be controlled by the present invention; and

FIG. 8 is an isometric view of a possible eye glasses with lenses that can be controlled by the present invention.

### DETAILED DESCRIPTION THE INVENTION

As stated herein, there remains a need for new methods and systems for tracking eye movement. The present inventors have invented new methods and systems for tracking ocular vergence movements (e.g., convergence or divergence) as a person changes his or her gaze by focusing at different distances, such as near viewing distances (about 40 cm), intermediate viewing distances (about 60 cm), and far viewing distances (about 400 cm). Thus, with the present methods and systems, it is now possible to detect neuromuscular activity associated with different visual task distances. In addition, the accuracy of predicting the visual task, or predicting the viewing distance based on the ocular vergence is high. For example, with the present methods and systems, the visual task prediction accuracy is at least 70%. In some embodiments, the visual task prediction accuracy is at least 80%. In yet further embodiments, including the preferred embodiments, the visual task prediction accuracy is at least 90%.

The present invention makes use of stretchable electrodes, such as electrodes of the type disclosed in U.S. Patent Publication 2015/0380355 of Rogers et al., the completed contents of which is herein incorporated by reference. These electrodes are characterized by stretchable metallic or semiconducting structures with soft, elastomeric materials in a configuration allowing for elastic deformations to occur in a repeatable and well-defined manner. The electrodes provide a skin-like system for monitoring muscle movements or electrical signals characteristic of eye focusing. These types of electrodes, as used herein, are referred to as “skin-like electrodes” or “flexible skin-like electrodes”. Thus, it can be understood that when the phrases “flexible skin-like electrodes” or “skin-like electrodes” is used in the present application, these electrodes are also stretchable. With these electrooculogram recording skin-like electrodes, it is now possible to record biopotentials associated with ocular vergence movements without the use of sticky adhesives that or messy conductive pastes that are associated with existing EOG recording electrodes, and which may cause pain or discomfort with long term use. In addition, in view of the present invention, it is now possible to record and track ocular vergence movements without the use of implantable inductive coils, which require implantation behind the eye of a person and require a large immobile magnetic reader. With the present methods and systems, EOG recordings of ocular vergence movements can be obtained at a higher resolution than previously possible, and that is required to detect or extract one degree of motion differences.

The flexible skin-like electrodes used herein can be made using conventional microlithography techniques, as understood by persons of skill in the art. As an example, a primer layer can be formed by spin coating, and a sacrificial layer can be formed on the primer layer, and a polyimide layer can be formed on the sacrificial layer. A conductive metal, such as gold, silver, or copper, can be deposited on the polyimide layer using a suitable technique, such as plasma sputtering. A photolithographic wet etching process can then be used to etch the material, and a reactive ion etcher can remove the polyimide layer around the patterned metal layer. The sacrificial layer is then removed, and the electrode pattern can be transferred to a thin elastomer layer.

The flexible skin-like electrodes are sufficiently flexible and stretchable to conform to the shape of the skin surface to which they are applied. For example, the electrodes can be reliably secured to irregularly shaped body structures, such as the nose, cheeks, around the eyes, or on the forehead. In addition, the flexible skin-like electrodes are relatively thin so that they don't interfere significantly with a person's normal activities. In some embodiments, the flexible skin-like electrodes have a maximum thickness less than about 100 micrometers. For example, the flexible skin-like electrode may have a maximum thickness from 0.1 micrometers to 100 micrometers. In further embodiments, the maximum thickness of the electrodes is less than 50 micrometers. In still further embodiments, the maximum thickness is less than 10 micrometers. In addition, the electrodes may have a relatively constant thickness such that the thickness does not vary by more than 20% from the maximum thickness. The present flexible skin-like electrodes also have a relatively low Young's modulus. In some embodiments, the flexible skin-like electrodes may have a modulus of less than about 1 MPa. For example, the flexible skin-like electrodes may have a modulus from 0.1 MPa to 1 MPa. In further embodiments, the electrodes may have a modulus less than 0.5 MPa. In additional embodiments, the electrodes may have a modulus less than 0.2 MPa.

It some embodiments of the present methods and systems, it is desirable to provide flexible skin-like electrodes that are difficult to see when placed on a person's skin. Thus, in some embodiments, the flexible skin-like electrodes have a transparency of at least 60% (i.e., at least 60% of visible light is transmitted through the electrode when examined with a light transmittance meter). In further embodiments, the flexible skin-like electrodes have a transparency of at least 80%. In additional embodiments, the flexible skin-like electrodes have a transparence of at least 85%. Furthermore, although some embodiments of the present methods and systems utilize flexible skin-like electrodes having opaque metals, such as gold, other embodiments may use transparent materials, such as graphene silver wires or nanowires, for the electrodes, the interconnects, or both.

Accordingly, in one aspect, a method of tracking ocular vergence movement of an eye of a person includes a step of providing a plurality of flexible skin-like electrodes configured for placement on a person's head. The method also includes recording electrooculogram data with the plurality of electrodes and a data acquisition unit, and transmitting data from the data acquisition unit to a computer configured to determine ocular vergence from the data received from the data acquisition unit.

The flexible skin-like electrodes are sufficiently flexible and thin that they can be applied at various locations on a person's face or head. It may be desirable to either configure the electrodes so that are not easily seen, or it may be desirable to place the electrodes in locations that are difficult to see by other people, for example, it may be possible to place one or more electrodes behind an ear, under a hairline, or at a base of a person's neck. In some embodiments, the electrodes are configured to be placed in a location where the electrodes can detect the activity of the medial recti extraocular muscles of the right and left eyes. As will be discussed herein, these locations may be appropriate when tracking the vergence movements at near viewing distances and intermediate viewing distances as contraction of both of these muscles is a requirement for near and intermediate vision, regardless of gaze location. Alternatively, in some embodiments, including the experimental embodiments described herein, the electrodes can be placed on the nose, near the eyes, and on the forehead of a person. The electrodes of the present methods and systems can be configured to be worn for a few hours, at least one day, at least one week, or possibly longer without irritating the skin of the wearer.

As described herein, two or more electrodes are used to track the ocular vergence movements of an eye. In some embodiments of the present methods and systems, the ocular vergence movements are recorded in one eye; and in other embodiments, the ocular vergence movements are recorded in both eyes. In some embodiments of the present methods and systems, including the experimental embodiments described herein, the methods and system use five to seven flexible skin-like electrodes. Although more electrodes may provide improvements in recording or detecting accuracy, it is possible to achieve high levels of accuracy with fewer electrodes by adjusting the data analytical parameters, such as filtering and classification parameters.

The electrooculogram can be recorded with the flexible skin-like electrodes and stored in a data acquisition unit. The data acquisition unit is coupled to the electrodes via a wired connection or a wireless connection. The data acquisition unit has a memory to store the electrooculogram recordings that it receives from the electrodes, and it has one or more transmitters to transmit the data to a computer for further processing. Any suitable communication protocol can be used to allow the data acquisition unit to receive and transmit the data. As described herein, in the experimental embodiments, the data acquisition unit used a Bluetooth communication protocol.

As discussed herein, the electrooculogram data is processed to filter out noise from the recorded signals, and is further processed to classify whether the signal properties correspond to a near viewing distance, an intermediate viewing distance, or a far viewing distance. In some embodiments, the processing of the data is done by the data acquisition unit. In other embodiments, the processing is done by a computer, such as a desktop computer, a laptop computer, a tablet computer, or a smartphone. As used herein, a computer refers to a device that has one or more processors capable of performing functions to classify the recorded signals. In yet other embodiments, some of the processing of the data is done by the data acquisition unit, and some of the processing of the data is done by the computer.

In some embodiments of the present methods, an additional step of generating a notification of an eye position is provided. The notification can be a visual signal that is perceptible by a person. Or, the notification can be a signal that is perceptible by a device. Depending on the particular method, the signal can be used to cause a change in visual properties of an ophthalmic device. For example, a signal can be used to cause a change in the focal length of an ophthalmic lens, such as a spectacle lens, a contact lens, or an intraocular lens. Or, a signal can be used to cause a change in a visual display of a head-mounted device that has a stereoscopic visual display, such as a virtual reality headset or an augmented reality headset.

Some embodiments of the present methods may include a step of determining whether the person is focused at a near distance, an intermediate distance, or a far distance based on the data received from the data acquisition unit. As used herein, near distance, intermediate distance, and far distance have their ordinary meaning in the context of opticians or eye care practitioners. More objectively, typically, a near distance or near viewing distance is about 40 cm, an intermediate distance is about 60 cm, and a far distance is about 400 cm.

Additional embodiments of the present methods may include a step of classifying the ocular vergence movement of the eye at an eye motion of less than five degrees. As stated herein, with the present methods and systems, high resolution electrooculogram recordings can be obtained and processed. The high resolution is necessary for these ocular vergence movements. With the present methods and systems, it is possible to extract as little as one degree of motion of the eye. Whereas conventional electrodes and existing methods only allow for measurement of at least five degrees of motion, the present systems provide for much finer recording resolution. In some embodiments, the methods and systems enable the classification to occur at an eye motion of between one and three degrees.

As alluded to above, the present methods and systems can be configured to provide one or more output signals. Accordingly, some embodiments of the present methods comprise a step of generating an output signal based on the ocular vergence of the eye or eyes. The output signal is effective in causing a visual change in an ophthalmic device. As used herein, an ophthalmic device refers to a device that interacts with a person's eye or eyes to provide a visual effect. For example, an ophthalmic device can be a vision correcting device, such as spectacles, a contact lens, or an intraocular lens. Or, an ophthalmic device can be a device held or worn by a person that has a visual display. In some embodiments, the ophthalmic device is a head-mountable device comprising a stereoscopic visual display, such as a virtual reality headset or augmented reality headset. In some embodiments, the methods include a step of changing a focal length of an ophthalmic lens with the output signal so that the ophthalmic device provides clear visual acuity to a person having the ophthalmic device located near the person's eye at different viewing distances. For example, the output signal can cause a change in the focal length of a spectacle lens, a contact lens, or an intraocular lens.

In some further embodiments of the present methods disclosed herein, an additional step is provided. Such embodiments include a step of measuring electrical activity associated with medial recti muscle contractions, pupil contractions, or ciliary muscle contractions, or combinations thereof. These additional measuring steps are in addition to the measurement of the ocular vergence movement. With such additional steps, the accuracy of predicting the visual task distance can be improved.

Accordingly, in view of the disclosure herein, it can be appreciated that another aspect of the present invention relates to systems for tracking ocular vergence movement of an eye of a person. These systems include a plurality of flexible skin-like electrodes, as described herein; a data acquisition unit in communication with the plurality of electrodes, in which the data acquisition unit is configured to receive electrooculogram data from the plurality of electrodes; and a computer readable medium configured to determine ocular vergence from data received from the data acquisition unit.

The computer readable medium, as used herein, can be located on a computer, or it can be located on a removable media storage device, such as a disk, a flash drive, or the like. The computer readable medium includes software configured to analyze and classify the data recorded by the electrodes and the data acquisition unit. The classification by the software can be performed by one or more algorithms as described herein in relation to the experimental embodiments. Thus, it can be understood that embodiments of the present systems include a kit that includes a plurality of skin-like electrodes, a data acquisition unit, and software for analyzing the recorded electrooculogram data, which software can be installed on a computer of a person's choice.

As discussed herein, the data acquisition unit may be a wireless device, or it may be a wired device. Preferably, the data acquisition unit is a wireless device so that it receives and transmits data using any suitable wireless communication protocol, such as Bluetooth protocols and the like.

Some embodiments of the present systems further include a computer. And yet further embodiments may include a computer having the computer readable medium.

Any of the preceding systems may also include an ophthalmic device configured to receive an output signal based on the ocular vergence movement that has been recorded. In some embodiments, the ophthalmic device is an ophthalmic lens, such as a spectacle lens, a contact lens, or an intraocular lens. Or, the ophthalmic device may be a head-mountable device comprising a stereoscopic visual display, as described herein.

In view of the disclosure herein, another aspect of the present invention relates to methods of controlling the focal length of an ophthalmic device, including the steps of providing an ophthalmic device that includes an electrically tunable optic. The electrically tunable optic is configured to change the focal length of the ophthalmic device by causing the electrically tunable optic to switch from a first refractive power to a different second refractive power. The method also includes providing an electrode assembly that includes a plurality of flexible skin-like electrodes for placement on a person's head, and a data acquisition unit. The electrode assembly is configured to record electrooculogram data of the person. And, the method includes providing a computer readable medium configured to process the electrooculogram data recorded by the assembly to produce a processed signal corresponding to the ocular vergence movement of an eye of the person. The processed signal is effective to cause a change of the focal length of the ophthalmic device from the first refractive power to the second refractive power so that a person using the ophthalmic device has an acceptable visual acuity.

Another aspect of the present invention relates to a system for controlling the focal length of an ophthalmic device, that includes an ophthalmic device that has an electrically tunable optic configured to change the focal length of the ophthalmic device by causing the electrically tunable optic to switch from a first refractive power to a different second refractive power; an electrode assembly including a plurality of flexible skin-like electrodes for placement on a person's head, and a data acquisition unit, in which the electrode assembly is configured to record electrooculogram data of the person; and a computer readable medium configured to process the electrooculogram data recorded by the assembly to produce a processed signal corresponding to ocular vergence movement of an eye of the person, in which the processed signal is effective to cause a change of the focal length of the ophthalmic device from the first refractive power to the second refractive power.

In additional embodiments of the present methods, an additional step can include placing the flexible skin-like electrodes on the skin of a person's head.

Thus, with the present methods and systems, integration of flexible skin-like electrodes with a wireless telemetric device has been achieved.

Additional details of the various methods and systems of the present invention can be appreciated from the following detailed description of experimental embodiments of the present methods and systems.

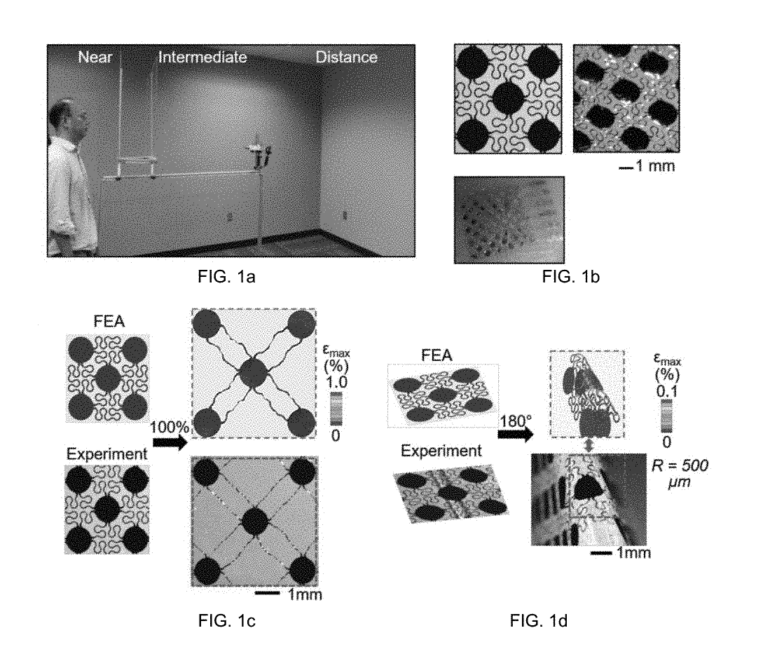

As mentioned herein, ocular experts consider recording vergence motions with electrooculograms to be difficult because of the required high resolution. Conventional gel electrodes are not as good at adhering to the skin as the soft skin-like electrodes disclosed herein, but another challenge is having a genuine experimental setup that can demonstrate vergence motions from test subjects. FIG. 1a shows a setup with distances of near, intermediate, and distant viewing distances. We have demonstrated a high classification accuracy using a large training dataset of electrooculography (EOG) vergence motions from our test setup in FIG. 1a. Utilizing the skin-like electrodes in FIG. 1b, we can observe the skin-like electrodes conforming to the skin in a small form factor. Finite element methods can validate the stretchability of the electrode up to 100% in the elastic region of the gold material, shown in FIG. 1c. The miniscule 1% maximum principal strain for stretching and 0.1% maximum principal strain from 180° bending is proof of the low stresses. Experimental validation in FIG. 1d is based on a 500 micron bending radius.

In these methods, the electrooculograms (EOG) recording utilizes a five-electrode setup, one electrode above and one electrode below one eye, one electrode on the outer canthi of each of the left and right eye, and the ground electrode on the forehead. This bipolar setup enables recordings for eye movements. Observing vergence motions requires the electrodes to be positioned at locations to achieve high resolution recordings and accommodate low degrees of eye motion.

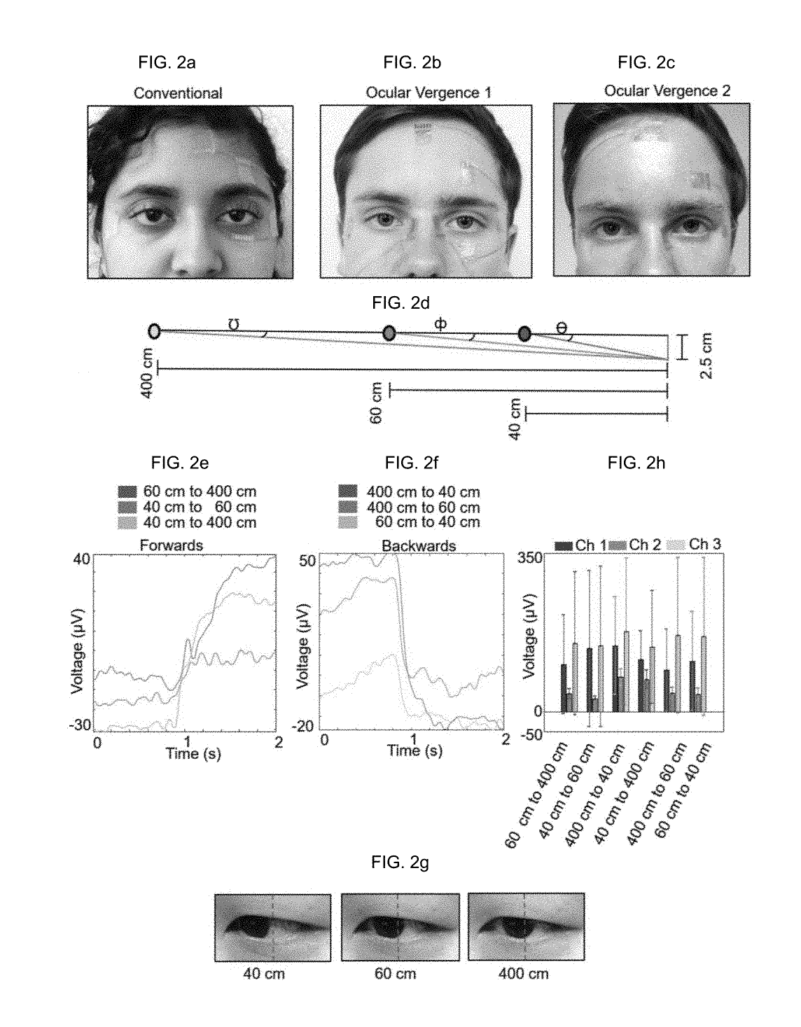

Unlike conventional EOG electrode positioning that depends upon eye movement in one direction, which is acceptable for a large range of potentials, the resolution of the flexible skin-like electrodes used in the present methods and systems is approximately 11 μV/°. FIG. 2a illustrates a conventional EOG electrode positioning system for vertical and horizontal movements. This bipolar setup does not work well for ocular vergence because of the reference electrodes for each channel. In order to preserve the signal quality of ocular vergence motions, a bipolar electrode setup was established using seven electrodes, as shown in FIG. 2b. In another some embodiments of the present methods and systems, five electrodes may be utilized, and may be configured for placement as shown in FIG. 2c.

Ocular vergence and electrooculography have a relationship which can benefit from optimization of electrograph recording parameters to have the maximum functionalities. In some experiments, a series of viewing distances were assessed with eye vergence to establish a metric for classification. Common distances humans observe in daily life were used as the basis for that metric in classification, as shown in FIG. 2d. The discrepancy of the degrees of eye motion is a necessary physical attribute for vergence classification which leads to higher accuracies with our classifier. FIG. 2e is an example of forward motions, near viewing distance (40 cm), intermediate viewing distance (60 cm), and far viewing distance (400 cm), corresponding to higher potentials as the degree of eye vergence (convergence) increases. Backwards eye vergence movements (divergence) have a similar pattern in the negative direction, FIG. 2f. Physically, the pupil moves away from the nose resulting in divergence recordings while the opposite occurs during convergence, FIG. 2g. Due to the temporal characteristics of the vergence motions, voltage potentials arising from head and apparatus placement causes typical mean and standard deviations of (140.17−112.06) μV±(173.40−109.85) μV, (105.25−92.24) μV+(110.09−90.66) μV, and (146.70−116.23) μV±(108.78−62.76) μV for reciprocal distances of 40 cm to 60 cm, 60 cm to 400 cm, and 400 cm to 40 cm. These values are shown in Table 1 and FIG. 2h for the center location and are subject to large variations because of the variability from person to person.

TABLE 1

| | Channels | | |
|---|---|---|---|
| Classes | #1 | #2 | #3 |
| 60 to 400 cm | 105.25 ± 110.09 | 39.69 ± 11.00 | 151.86 ± 158.43 |
| 40 to 60 cm | 140.17 ± 173.40 | 29.49 ± 4.49 | 145.43 ± 177.84 |
| 400 to 40 cm | 146.70 ± 108.78 | 76.75 ± 18.60 | 177.59 ± 162.94 |
| 40 to 400 cm | 116.23 ± 62.76 | 72.04 ± 19.73 | 143.06 ± 125.57 |
| 400 to 60 cm | 92.24 ± 90.66 | 41.47 ± 11.64 | 169.39 ± 172.82 |
| 60 cm to 40 cm | 112.06 ± 109.85 | 38.19 ± 13.94 | 166.83 ± 175.31 |

The acquired signals from vergence motions can be classified using a mathematical translation using statistical analysis. A wrapper feature selection algorithm was applied to determine if the utilization of ten features was optimized for our dataset. The classification algorithm includes the following features: definite integral, amplitude, velocity, signal mean, wavelet energy, Ctrapz, V (velocity), RMS (root mean square), Peak2RMS, and Peak2Peak. These features can be easily converted into a computer programming language, such as C, using a Matlab coder. All of the following features use a sliding window identified by the indices t₁ and t₂.

$$\begin{matrix}Definite integral = {\int }_{{t}_{1}}^{{t}_{2}} f ( t ) dt & ( 1 ) \\ Amplitude = {A}_{{t}_{1}} - {A}_{{t}_{2}} , where A = ( \frac{( f ( t ) )}{dt} ) & ( 2 ) \\ Velocity = \frac{{A}_{{t}_{1}} - {A}_{{t}_{2}}}{{t}_{1} - {t}_{2}} & ( 3 ) \\ Signal mean = \frac{1}{N} {\sum }_{{t}_{1}}^{{t}_{2}} f ( t ) , where N = number of samples & ( 4 ) \\ Wavelet energy = \sum \vert {C}_{a , b} * {C}_{a , b} \vert , \\ where {C}_{a , b} = {\int }_{{t}_{1}}^{{t}_{2}} f ( t ) {ϕ}_{a , b} ( t ) dt & ( 5 ) \\ Ctrapz = {\sum }_{i = 1}^{1000} {\int }_{t}^{i = 1} {f}_{i} ( t ) dt & ( 6 ) \\ V = \frac{1}{1000 - 1} {\sum }_{i = 1}^{1000} {\vert f ( t ) - μ \vert}^{2} & ( 7 ) \\ RMS = \sqrt{\frac{1}{1000} {\sum }_{i = 1}^{1000} {\vert fi ( t ) \vert}^{2}} & ( 8 ) \\ Peak 2 RMS = \frac{\vert max ( f ( t ) ) \vert}{RMS} & ( 9 ) \\ Peak 2 Peak = \frac{\vert max ( f ( t ) ) \vert}{\vert min ( f ( t ) ) \vert} & ( 10 )\end{matrix}$$

The area under the curve of the filtered signal (Eq. 1) can be determined using the trapezoidal method. The signal amplitude (Eq. 2) can be determined from the derivative filtered signal in which

### Aₜ_{₁}

and

### Aₜ_{₂}

are two consecutive peaks surpassing a threshold. A difference between the two peaks explains the order of the positive and negative peaks for each channel. The velocity (Eq. 3) is the amplitude difference divided by the time difference, and signal mean (Eq. 4) is the average of the filtered signals. The Haar wavelet energy transform (Eq. 5) outputs a set of scaled coefficients (C_{a,b}). The absolute multiplication of the coefficients creates the scalogram matrix, which is summed to receive the desirable wavelet energy feature. The sixth feature (Eq. 6) shows cumulative trapezoidal numerical integration in which the filtered signal f(t) is summed upon each unit step, i to i+1, using the trapezoidal method for quick computation. The next feature (Eq. 7) is the variance of the filtered signal. A root mean square (RMS) can be also used, (Eq. 3), in conjunction to the peak to root mean square, (Eq. 9). Together, a precise average and ratio assist the classifier in determining the class. The final feature is a ratio of the maximum over the minimum of the filtered signal window, (Eq. 10).

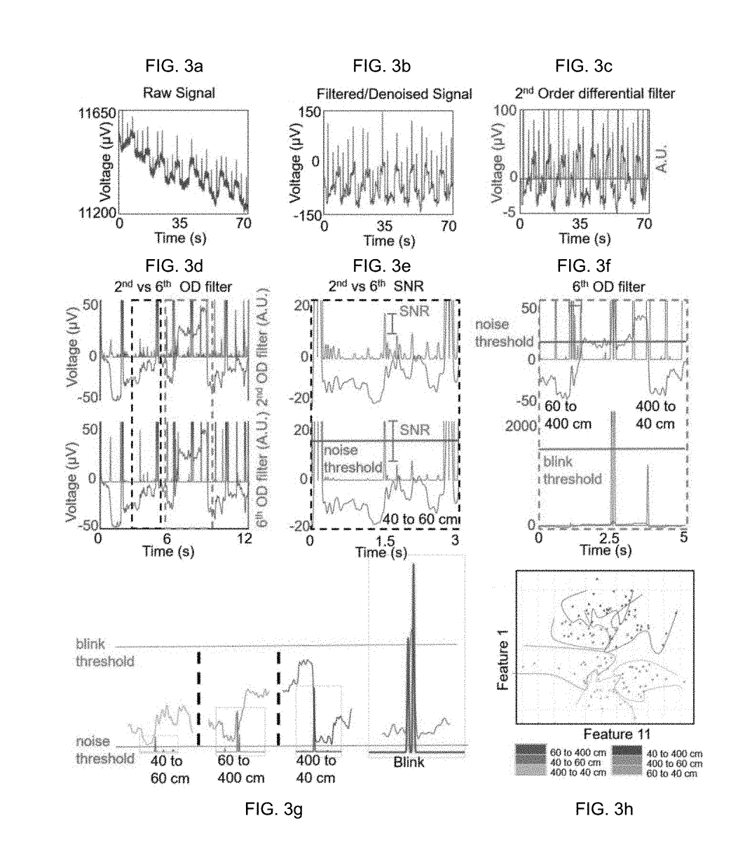

In order to further increase accuracy of the feature set with the classifier, and to incorporate it in real-time, a logic based algorithm can be applied. Prior to the algorithm implementation, the incoming data, presented in FIG. 3a, can be acquired via Bluetooth telemetry and preprocessed with a 3^{rd} order Butterworth bandpass filter, as shown in FIG. 3b. Subsequently, a two point differentiation can be applied on the filtered dataset raised to the 2nd power, as shown in FIG. 3c. In order to increase the discrepancy between smaller and larger peaks, the derivative filter signal can be raised to the 6th power, a comparison is presented in FIG. 3d. This algorithm applies multiple cases initiated by thresholds. The threshold is applied to the 6th power derivative filter for blinks, eye movements, and vergence motions. FIG. 3e shows the signal to noise ratio (SNR) comparison between the two derivative filters, as the signal becomes significantly larger than the noise surpassing the “noise threshold”. Removing noise from classification is important but we need to ensure that blinks and large eye movements are separated using a “blink threshold”. This number is sufficiently larger than the high amplitude vergence motions from 40 cm to 400 cm and 400 cm to 40 cm, as shown in FIG. 3f. Large movements become classified as blinks and eye movements, whilst small movements are separated into a window of vergence motions, as shown in FIG. 3g. Finally, the window can be transferred to the random subspace discriminant classifier that separates the dataset into its individual classes near, intermediate, and distance using an ensemble of classifiers, shown in FIG. 3h.

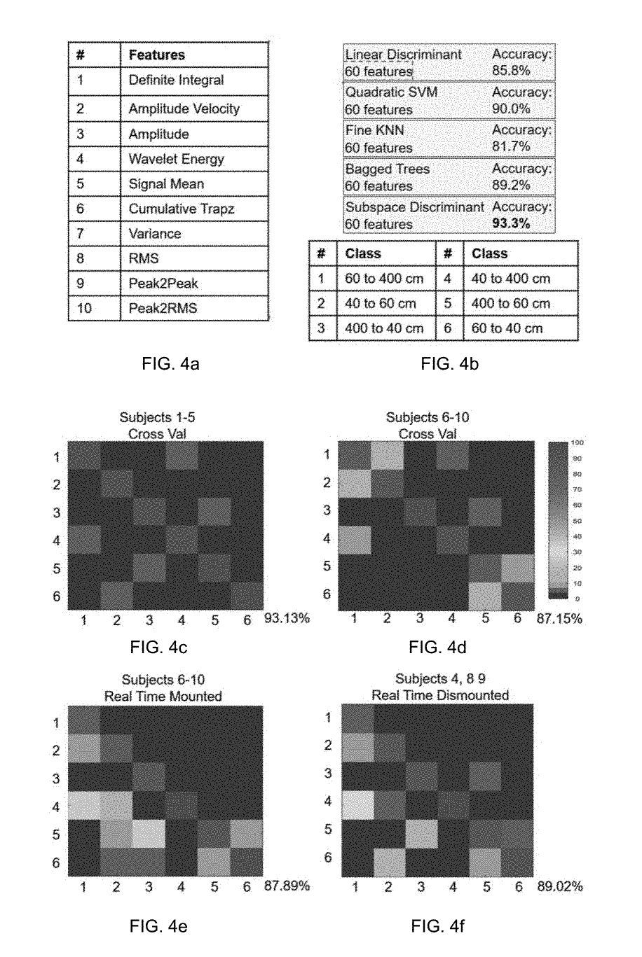

In order to select the best classifier for our purpose, we utilized Matlab's classification learner application. This application extrapolates a saved dataset by applying k-fold cross validation using the aforementioned features, as shown in FIG. 4a. Thirty features can be extracted using ten statistical measurements from three separate channels. Additionally, thirty more features can be extracted from the derivative filter signal, providing a total of up to sixty features for higher accuracy. We applied multiple classifiers with five-fold validation, as shown in FIG. 4b. The datasets from in vivo test subjects indicated a couple of classifiers, quadratic support vector machine and ensemble subspace discriminant, were consistently more accurate than other classifiers. The latter is consistently higher in accuracy with various test subjects in cross validation assessments. The ensemble classifier utilizes a random subspace with discriminant classification rather than nearest neighbor. Unlike the other ensemble classifiers, random subspace, as used herein, does not use decision trees. The discriminant classification combines the best of the feature set and discriminant classifiers while removing the weak decision trees to yield its high accuracy.

Real-time classification benefits from data validation prior to model implementation with an interface. Therefore we enlisted ten test subjects, and we measured vergence motions of the subjects' eyes over a two hour period. In this two hour period, the subjects were required to observe nine positions, center, 0, 45, 90, 135, 180, 215, 270, and 315 degrees. A total of 144 vergence motions were gathered for each position to ensure high cross validation accuracy. A verbal feedback stating the three commands “Near”, “Intermediate”, and “Distance” was used to train the classifier. The user responded to the commands and a window was recorded following the command. The first five subjects used the seven electrode EOG setup which yielded cross validation accuracies great than 93% as shown in confusion matrix in FIG. 4c. Using seven electrodes can be less desirable than fewer electrodes for the test subjects and for the test mediator, therefore systems and methods were developed using five electrodes, and still obtained accurate measurements. FIG. 4d presents the cross validation accuracy greater than 87% for the latter five test subjects with the five electrodes. When the five electrode system is implemented in the real-time classification test, the accuracy of the results ranged from 79% to 94% with an average of about 88%, as shown in FIG. 4e.

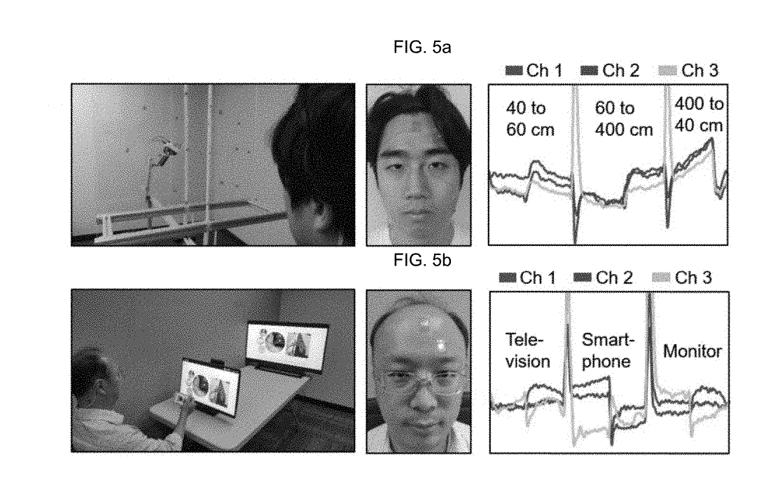

All the aforementioned tests were trained and tested upon using an optometric head stand. Therefore, another test was considered where three of the users dismount from the headstand and continued to retrain and retest all nine positions. FIG. 4f shows the real-time classification results from the dismounted test subjects with average accuracies of about 89%. Finally, experiments were conducted as a comparison study of activity recognition. Using the physical test apparatus of FIG. 5a, it was concluded that an accuracy of about 90% is feasible with vergence motions. Therefore, a vergence test with three common electronic items, smartphone (near), monitor (intermediate), and television (distance), was conducted using the test setup in FIG. 5b. The signal comparison from the test shows similarities in the horizontal direction with channel 1 and channel 2 but the vertical motion from channel 3 is more closely related to the vergence motions of the 270° position. Ultimately, the integrative device demonstrated versatility with eye vergence motions, electrooculograms (EOGs), and a novel machine learning algorithm.

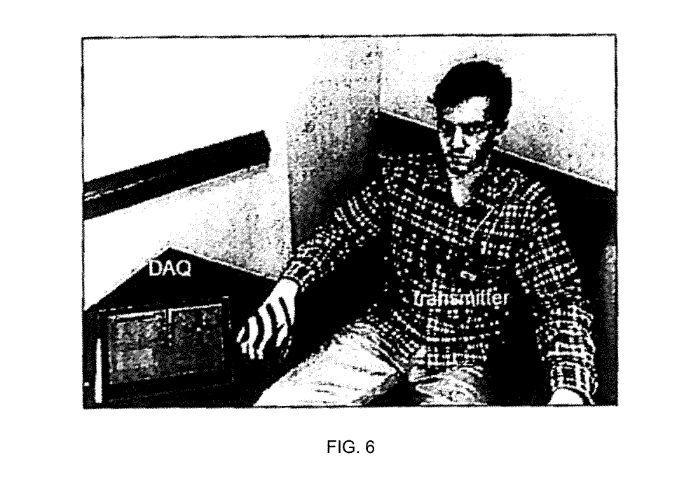

FIG. 6 shows a laboratory setup with a subject having electrodes attached to the skin around his eyes. In this example, the electrodes were connected by wires to a Bluetooth transmitter, here shown suspended by an attachment to the subject's shirt pocket. The Bluetooth transmitter serves as a data acquisition unit and may perform some signal processing and filtering before transmitting signals to the tablet which is programmed to determine eye vergence. The data can be transmitted to a tablet computer. The output of the tablet computer can then be transmitted to an electronic ophthalmic device in the form of a contact lens of wearable glasses. The electronic ophthalmic device is configured to change its focal length by causing the electrical tunable optic to switch from one refractive power to a different refractive power depending on the detected eye vergence as, for example, switching from near to medium or distant vergence.

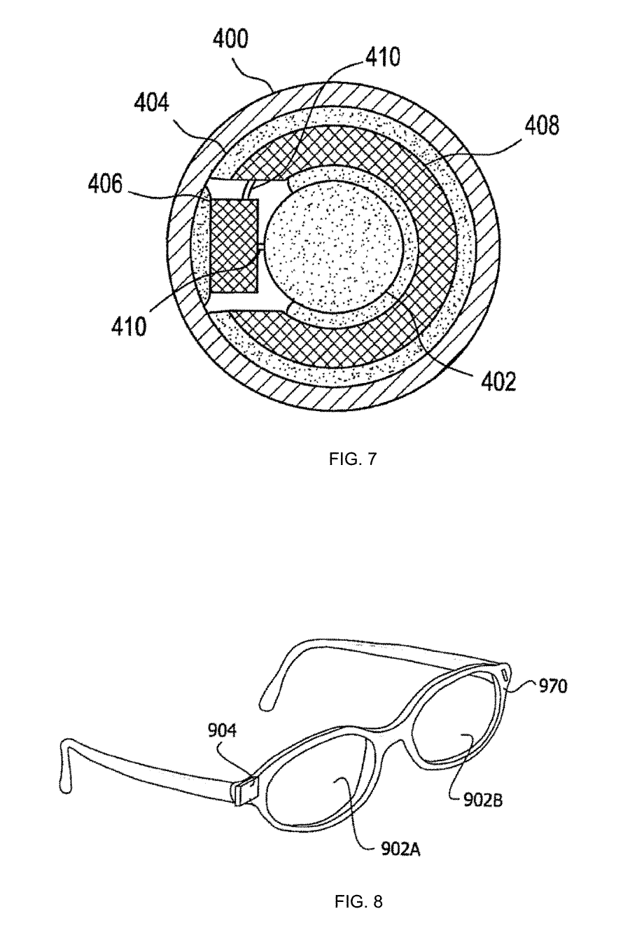

FIG. 7 is an illustration of one possible variable-optic electronic ophthalmic contact lens that may be used with the present invention. This lens is disclosed in detail in U.S. Patent Application Publication No. 2014/0022505 of Pugh et al. FIG. 8 is an illustration of one possible eyeglass system with adaptive lenses that may be used with the invention. This eyeglass system is disclosed in detail in U.S. Pat. No. 8,587,734 to Li. The complete contents of both patent applications are herein incorporated by reference.

Before the present invention, an eye vergence recording and classification technique using skin-like electrodes that enabled sensitive biopotential readings was not known. In prior methods and systems, the signals that were usually acquired from the eyes are eye movements, not eye vergence movements. Typical eye movement classification operates using more than 5° of eye motion. The eye vergence movements measured herein are classified in a range from 1° to 3°, and they are also contradictory to each other with a conventional electrode setup so the delta of the signal equivocates to zero. Utilizing an electrode system with five or seven electrodes, such as described herein, resolved this issue. Both the five electrode configuration and the seven electrode configuration produced accurate readings. After cross validation and real-time tests, the accuracies yielded about 94% accuracy. Activity recognition purposes were validated using a smartphone, monitor and television setup enabling eye vergence tracking using the present electrodes, vergence setup, and classification technique.

1. A method of tracking ocular vergence movement of an eye of a person, comprising:
providing a plurality of flexible skin-like electrodes configured for placement on a person's head;
recording electrooculogram data with the plurality of electrodes and a data acquisition unit; and
transmitting data from the data acquisition unit to a computer configured to determine ocular vergence from the data received from the data acquisition unit.

2. The method of claim 1, further comprising generating a notification of an eye position based on the recorded electrooculogram data.

3. The method of claim 1, wherein the method comprises providing from about 5 to about 7 flexible skin-like electrodes configured for placement on the skin of the person.

4. The method of claim 1, further comprising determining whether the person is focused at a near distance, intermediate distance, or far distance based on the data received from the data acquisition unit.

5. The method of claim 1, wherein the data is wirelessly transmitted from the data acquisition unit to the computer.

6. The method of claim 1, further comprising classifying ocular vergence movement of the eye at an eye motion of less than 5 degrees.

7. The method of claim 1, further comprising generating an output signal based on the ocular vergence, the output signal is effective to cause a visual change in an ophthalmic device.

8. The method of claim 7, further comprising changing a focal length of an ophthalmic lens with the output signal so that the ophthalmic device can provide clear visual acuity to a person having the ophthalmic device located near the person's eye at different viewing distances.

9. The method of claim 8, wherein the ophthalmic lens is a spectacle lens, a contact lens, or an intraocular lens.

10. The method of claim 7, wherein the ophthalmic device is a head-mounted device comprising a stereoscopic visual display.

11. A system for tracking ocular vergence movement of an eye of a person, comprising:
a plurality of flexible skin-like electrodes configured for placement on a person's head;
a data acquisition unit in communication with the plurality of electrodes to receive recorded electrooculogram data from the plurality of electrodes; and
a computer readable medium configured to determine ocular vergence from data received from the data acquisition unit.

12. The system of claim 11, wherein the data acquisition unit is a wireless device.

13. The system of claim 11, further comprising a computer, and the computer readable medium is provided on the computer.

14. The system of claim 11, further comprising an ophthalmic device configured to receive an output signal based on the ocular vergence movement.

15. The system of claim 14, wherein the ophthalmic device is an ophthalmic lens.

16. The system of claim 14, wherein the ophthalmic device is a head-mountable device comprising a stereoscopic visual display.

17. A method of controlling the focal length of an ophthalmic device, comprising the steps of:
providing an ophthalmic device comprising an electrically tunable optic configured to change the focal length of the ophthalmic device by causing the electrically tunable optic to switch from a first refractive power to a different second refractive power;
providing an electrode assembly comprising a plurality of flexible skin-like electrodes for placement on a person's head, and a data acquisition unit, the electrode assembly configured to record electrooculogram data of the person; and
providing a computer readable medium configured to process the electrooculogram data recorded by the assembly to produce a processed signal corresponding to ocular vergence movement of an eye of the person; the processed signal being effective to cause a change of the focal length of the ophthalmic device from the first refractive power to the second refractive power so that a person using the ophthalmic device has an acceptable visual acuity as determined by the person.

18. A system for controlling the focal length of an ophthalmic device, comprising:
an ophthalmic device comprising an electrically tunable optic configured to change the focal length of the ophthalmic device by causing the electrically tunable optic to switch from a first refractive power to a different second refractive power;
an electrode assembly comprising a plurality of flexible skin-like electrodes for placement on a person's head, and a data acquisition unit, the electrode assembly configured to record electrooculogram data of the person; and
a computer readable medium configured to process the electrooculogram data recorded by the assembly to produce a processed signal corresponding to ocular vergence movement of an eye of the person; the processed signal being effective to cause a change of the focal length of the ophthalmic device from the first refractive power to the second refractive power.

19. A method for controlling the focal length of an electronic ophthalmic device, comprising the steps of:
providing an electronic ophthalmic device comprising an electrically tunable optic configured to change the focal length of the electronic ophthalmic device by causing the electrically tunable optic to switch from a first refractive power to a different second refractive power;
providing a thin-film electrode assembly comprising a plurality of flexible electrodes configured to detect electrical activity associated with visual task distance of a person using the electronic ophthalmic device;
processing a signal from the thin-film electrode assembly to produce a processed signal corresponding to the electrical activity associated with visual task distance detected by the thin-film electrode assembly; and
changing the focal length of the electronic ophthalmic device based on the processed signal and the predicted visual task distance so that the person using the electronic ophthalmic device has an acceptable visual acuity as determined by the person.

20. An ophthalmic system having an electronically controlled focal length, said system comprising:
an electronic ophthalmic device comprising an electrically tunable optic configured to change the focal length of the electronic ophthalmic device by causing the electrically tunable optic to switch from a first refractive power to a different second refractive power; and
a thin-film electrode assembly comprising a plurality of flexible electrodes configured to detect electrical activity associated with visual task distance of a person using the electronic ophthalmic device,
wherein the thin-film electrode assembly is configured to communicate with the electronically tunable optic to cause a change in the refractive power of the electrically tunable optic based on a signal corresponding to the electrical activity associated with visual task distance of the person using the electronic ophthalmic device.
